(19) United States
(12) Patent Application Publication
(10) Pub. No.: US 2018/0296390 A1
(43) Pub. Date: Oct. 18, 2018

(54) APPARATUS AND METHOD FOR TREATING A NUMBER OF EYE CONDITIONS OF A USER INCLUDING DRY EYE SYNDROME AND/OR BURNS TO THE EYES

(71) Applicant: David Hoare, Highett (AU)

(72) Inventor: David Hoare, Highett (AU)

(21) Appl. No.: 15/954,639

(22) Filed: Apr. 17, 2018

Related U.S. Application Data
(60) Provisional application No. 62/486,772, filed on Apr. 18, 2017.

(51) Int. Cl. A61F 9/00; A61F 9/02; A61F 7/00

(52) U.S. Cl. CPC A61F 9/0026; A61F 9/029; A61F 2007/0062; A61F 2007/0004; A61F 7/00

(57) ABSTRACT

Disclosed is an apparatus and method for treating a number of eye conditions of a user including dry eye syndrome or burns to the eyes. The apparatus include a pair of protective close fitting glasses flexibly connected to form a goggle to securely cover a pair of eyes of the user, a frame structure including input means is provided around the pair of protective close fitting glasses, one or more diffuser in fluid communication with the frame structure to provide heat, moisture content and the medications to the eyes of the user, and one or more mode selector means configured on the diffuser, and/or provided as a controller for selecting a desired operational mode for providing a desired amount of heat, moisture and the medication to the eyes of the user to treat the number of eye conditions including dry eye syndrome or burns to the eyes.

### CROSS REFERENCE TO RELATED APPLICATION

The present application claims the priority benefits of U.S. Provisional Application No. 62/486,772, filed on Apr. 18, 2017, titled “A pair of goggles used to treat dry eye syndrome by providing heat and moisture to the eyes. It uses a diffuser to deliver saline to the goggles.” which is incorporated herein by reference in its entirety.

### TECHNICAL FIELD OF INVENTION

The present invention relates generally to an apparatus, and method for treating dry and irritated eye conditions. More specifically, it relates to an apparatus and a method of altering the heat and moisture around the eyes of users to reduce the discomfort of dry eye syndrome and/or burns to the eyes.

### BACKGROUND

Dry eye syndrome is one of the most commonly treated eye problems in the world. Dry eye syndrome is also known as keratitis sicca, keratoconjunctivitis sicca (KCS) xerophthalmia, and lacrimal insufficiency. It is estimated that over thirty million persons worldwide suffer from severe dry eye conditions, e.g., Dry Eye Syndrome or Chronic Dry Eye Disease (CDED), both clinically known as keratoconjuctivitis sicca. CDED is a condition that results from inflammation of the eye, as well as inflammation of the tear-producing glands. This inflammation decreases the eyes' ability to produce natural tears, which help protect the eyes from irritation and keep them moisturized and lubricated. Persons who suffer from CDED often experience constant pain from eye irritation, including a sandy or gritty sensation that, if untreated, can lead to scarring or ulceration of the cornea, which can lead to a partial or total loss of vision.

Dry eye syndrome typically results from deficiency in the quality or quantity of tears produced by the patient. Precorneal tear film of eyes has traditionally been considered to have a three-layered structure. The closest to the cornea lies the mucin, or mucus layer. The mucin layer provides an interface between the corneal epithelium and the remainder of the tear film. Overlying the mucin layer is the watery aqueous layer, which is the thickest layer of the three. The outermost layer of the precorneal tear film is the lipid layer. The lipid layer is an oily film that reduces evaporation from the aqueous layer beneath it. The middle aqueous layer provides moisture to the corneal tissue, carries important nutrients, and serves to remove metabolic waste produced by the cornea. Deficiency in any of the three layers of the precorneal tear film can result in complaints of dry, gritty feeling or burning eyes. The mucin that forms the mucin layer, nearest to the cornea, is secreted by goblet cells in the conjunctiva. The conjunctiva is the transparent tissue that covers the sciera and the backside of the eyelids. The mucin layer functions to decrease surface tension of the tear film. In addition, the cornea itself is hydrophobic. Without the mucin layer to provide a bridge between the cornea and the aqueous layer, the aqueous layer would bead up and allow dry spot formation on the cornea. The aqueous layer is secreted primarily by the glands of Wolfring and Krause located in the eyelid margin. The aqueous layer helps provide an optically smooth, transparent surface to the precorneal tear film. The lipid layer is secreted by the meibomian glands, and the glands of Zeiss and Moll. The glands of Zeiss and Moll are also located at the eyelid margin.

Most cases of CDED result from normal aging of the eye, however, CDED or dry, irritated, tired eyes can occur at any age. It is estimated that nearly 75% of people over the age of 65 will experience CDED conditions. CDED occurs in both men and women, however, it is most common in women who are post-menopausal or pregnant. Approximately 30 million people, of which approximately 90% are women, have CDED as a consequence of Sjδgren's syndrome, which is a chronic, slowly progressive autoimmune disease characterized by dryness of the eyes and mouth and recurrent salivary enlargement. Other diseases, such as rheumatoid arthritis, lupus, scleroderma, and thyroid disease, can also cause CDED. In addition, health, environmental, and lifestyle conditions can also cause CDED. For example, people suffering from allergies and persons, who wear contact lenses, have a greater risk of developing CDED. Other circumstances that significantly dry, irritate, and fatigue the eye include the continuous computer use, low humidity environments, such as flying in an airplane, in which the relative humidity is typically between 5% to 15%, use of certain medications, after eye surgery and due to the usage of certain medical conditions, such as bletharitis i.e. the inflammation of the eyelids.

Blinking is essential to maintenance of the precorneal tear film. During each blink, the eyelid wipes over the surface of the cornea, smoothing the mucin layer and spreading the overlying aqueous and lipid layers to provide a completely wetted surface. In between blinks, the tear film thins due to evaporation of the aqueous layer. If evaporation is excessive, dry spots may form on the surface of the cornea. Deficiency, or imperfect quality, of any of the three component layers can lead to dry eye symptoms. Many systemic and external factors can contribute to dry eye syndrome. For example, Sjogren's syndrome is associated with arthritic diseases in combination with dry eye and dry mouth. Deficiency of Vitamin A, use of oral contraceptives and environmental factors can all contribute to dry eye syndrome. The adnexa of the eye may also be involved in dry eye syndrome. The adnexa of the eye include the structures surrounding the eye such as the eyelids, eyelashes, the tear drainage and tear production structures. Blepharitis commonly contributes to dry eye syndrome. Blepharitis typically results from bacterial infection of the tiny glands in the margin of the eyelid. These glands include the glands of Zeiss, Moll and Wolfring as well as the meibomian glands. Most commonly, the affected glands are the meibomian glands. In bacterial blepharitis, bacterial infection causes the meibomian glands to become plugged, and thus not be able to produce a normal lipid layer to contribute to the tear film. Some bacteria that infect the glands also secrete exotoxins that seep out of the glands into the eye and injure the corneal epithelium.

Treatments of dry eye syndrome vary depending upon the type of presentation. The most common treatment for dry eye syndrome is the use of artificial tear supplements to provide additional moisture and lubrication to the corneal surface. Artificial tear eye drops are placed on the eye by the patient. Artificial tear supplements must be used regularly and often to be effective. In some other cases, lubricant ointments are also be employed. Ointments are usually used at bedtime because they tend to be messy and blur vision. For some patients, even the use of ointments is not sufficient to provide comfort during sleep.

In some instances, one treatment for dry eye syndrome is to partially or completely close one or more lacrimal puncta to reduce tear outflow into the lacrimal drainage apparatus. Traditionally, this closure was accomplished surgically or by cautery. In the last decade, however, temporary and permanent punctal occlusion plugs have been utilized. Permanent punctal plugs are typically made from surgical silicone; temporary plugs are generally made of soluble collagen. Collagen plugs dissolve over a period of days and are helpful in diagnosis. Punctal plugs are placed into the lacrimal puncta, or lacrimal drainage ducts. The plugs impede the outflow of tears from the eye. This approach slows the outflow of tears and retains them in the eyes longer, often relieving symptoms. Punctal plugs have the distinct advantage of being readily removable and avoid the issues of scar formation.

Patients who have severe dry eye syndrome often suffer disrupted sleep because they cannot go for longer than an hour or so without applying tear supplements to the eyes. This can lead to pronounced sleep deprivation and a consequent reduction in quality of life. Different methods as stated above have been employed to treat the dry eye syndrome. For example, U.S. Pat. No. 9,724,230 discloses a method and apparatus for the treatment of dry eye syndrome using adhesive strips that is specifically contoured or shaped to adhere to selected regions around a patient's eyes. Similarly, U. S. Pat. No. 7,186,233 discloses a method for treating dry-eye syndrome includes collecting fluid from inside the eye; and transporting the collected fluid into the fornix of the eyelid such that with each blink, fluid accumulated in the fornix is spread over the eye.

Despite the many treatment options are available, there remains no cure for dry eye syndrome. Many patients still suffer from substantial and even debilitating discomfort because of dry eye syndrome. Very few treatment options exist to provide comfort for dry eye syndrome and burns to the eyes of patients during sleep. Thus there is a need for an apparatus, system and method for treating dry, burns to the eyes and irritated eye conditions.

### BRIEF SUMMARY

Before the present systems and methods, enablement are described, it is to be understood that this application is not limited to the particular systems, and methodologies described, as there can be multiple possible embodiments which are not expressly illustrated in the present disclosures. It is also to be understood that the terminology used in the description is for the purpose of describing the particular versions or embodiments only, and is not intended to limit the scope of the present application.

According to an aspect of the present invention, there is provided an apparatus for treating a plurality of eye conditions of a user including dry eye syndrome or burns to the eyes. The apparatus includes a pair of protective close fitting glasses flexibly connected to form a goggle to securely cover a pair of eyes of the user.

According to the same aspect, the apparatus includes a frame structure provided around the pair of protective close fitting glasses, wherein the frame structure includes at least one input means associated with the goggles for providing heat, moisture content and at least one medication to the eyes of the user.

According to the same aspect, the apparatus includes at least one diffuser in fluid communication with the frame structure to provide heat, moisture content and the at least one medication to the eyes of the user.

According to the same aspect, the apparatus further includes at least one mode selector means configured on at least one of the diffuser, and as a hand held controller for selecting at least one desired operational mode.

According to the same aspect, the apparatus enables the users to select a desired mode of operation for providing a desired amount of heat, moisture and the at least one medication to the eyes of the user to treat the plurality of eye conditions including dry eye syndrome or burns to the eyes.

According to another aspect of the present invention, provided is a method of treating a plurality of eye conditions of a user including dry eye syndrome or burns to the eyes. The method includes the steps of providing a goggle having a pair of protective close fitting glasses surrounded using a frame structure, wherein the frame structure includes at least one input means for receiving moisture content and at least one medication, wherein the frame structure includes a plurality of passages for providing moisture content and at least one medication received through the input means to the eyes of the user.

According to the same aspect, the method includes providing a diffuser having at least one fluid, at least one substance for providing moisture content and at least one medication.

According to the same aspect, the method includes the step of allowing the user to connect the diffuser to the input means of the frame structure using at least one flexible connecting means.

According to the same aspect, the method includes the step of allowing the users to wear the goggles and to select at least one mode of operation using at least one mode selector means provided configured on at least one the diffuser, and as a hand held controller.

According to the same aspect, the method further includes the step of connecting the diffuser to a power supply means for supplying moisture content and the at least one medication to the eyes of the user.

The method characterized in that the moisture content and the at least one medication provides heat and moisture content to the eyes of the user to treat the plurality of eye conditions including dry eye syndrome or burns to the eyes.

Additional objects and aspects of the present invention would appear and become clear as the detail description proceeds with the accompanying drawings.

### BRIEF DESCRIPTION OF THE DRAWINGS

The foregoing summary, as well as the following detailed description of preferred embodiments, is better understood when read in conjunction with the appended drawings. There is shown in the drawings example embodiments, however, the application is not limited to the specific system and method disclosed in the drawings.

FIG. 1 illustrates a simple diagrammatic representation of the arrangement of the present system or apparatus for providing heat, moisture content and medications to the eyes of the user and thereby treating a variety of eye conditions of the user including dry eye syndrome or burns to the eyes, according to a preferred embodiment of the present invention;

FIG. 2 illustrates an exploded view of the goggle, according to an embodiment of the present invention; and

FIG. 3 shows a flowchart showing the steps for providing heat and moisture content to the eyes of the user and for treating the eye conditions of a user including dry eye syndrome or burns to the eyes, according to a preferred embodiment of the present invention.

### DETAILED DESCRIPTION

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical, electrical and other changes may be made without departing from the scope of the embodiments. The following detailed description is therefore not to be taken in a limiting sense.

The present invention relates to a system and an apparatus for treating a variety of eye conditions common among people including dry eye syndrome, burns to the eyes. The present system and apparatus is also developed to provide comfort in form of desired amount of heat and moisture content around the eyes of the users. This helps to control the loss of natural tears generated by the glands associated with the eyes of the user. Also the control of heat and humidity or moisture content around the eyes of the user maintains the fluid present in the eyes of the user and thereby improves the overall health of the eyes of the user. The present apparatus can also be utilized to continuously provide specific medications to the eyes of the user. This helps in treating a variety of eye diseases and other eye conditions such as the dry eye syndrome associated with the users.

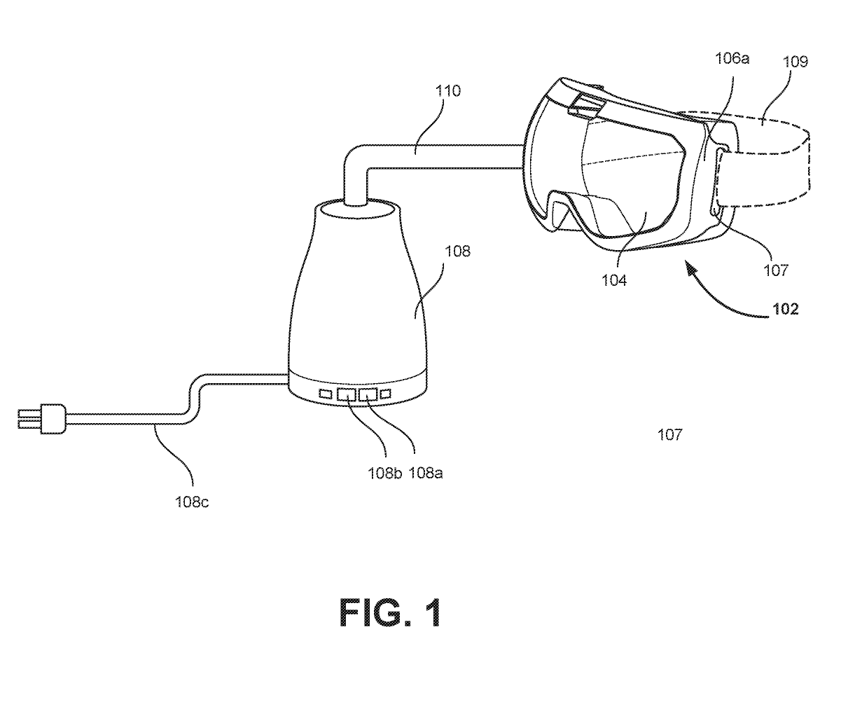

The various features and embodiments of the method, apparatus or system for treating the variety of eye diseases and other eye conditions such as the dry eye syndrome, burns to the eyes associated with the users will now be explained in conjunction with the description of FIGS. 1-3.

Referring to FIG.1, illustrates a simple diagrammatic representation of the arrangement of the present apparatus or system 100 for providing heat, moisture content and medications to the eyes of the user and thereby treating a variety of eye conditions of the user including dry eye syndrome, burns to the eyes. As shown, the apparatus or system 100 includes a goggle 102 to securely cover a pair of eyes of the user, and a diffuser 108 in fluid communication with the goggle 102 for delivering heat, moisture content and medications to the eyes of the user.

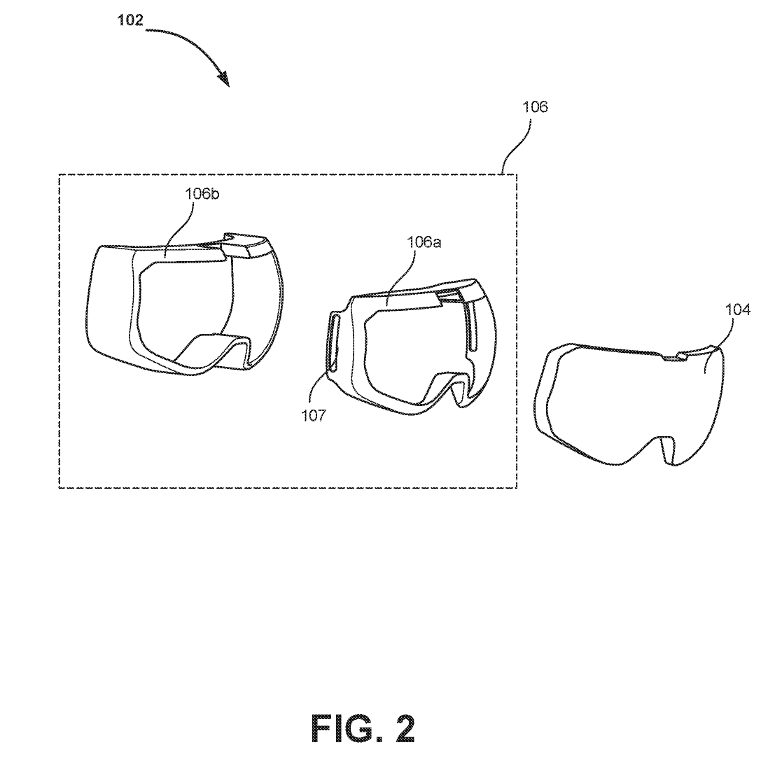

As shown in FIG.2, a pair of protective close fitting glasses 104 may be flexibly connected to form a goggle 102 to securely cover the pair of eyes of the user. The pair of close fitting glasses 104 is connected to a frame structure 106 (particularly to a first part 106a of the frame structure 106) using flexible central connector to form the goggles 102. A semi flexible frame structure 106 including the first part 106a, and a second part 106b is provided around the pair of protective close fitting glasses 104. The frame structure 106 attaches to the edges of the glasses 104 and forms the skeleton of the goggles 102. As seen, the frame structure 106 is formed of two parts 106a, 106b, wherein the part 106b of the frame structure 106 engages inside the groove (not shown) formed at the back of the part 106a. As seen, the part 106a of the frame structure 106 may also have provisions 107 on the two sides for attaching a strap 109 that may go over the user's head in order to hold the goggle 102 in place, when the goggle 102 is in use. According to some other embodiment, the frame structure 106 may be formed as a single piece structure.

In a preferred embodiment, the glasses 104 are made up of plastic or fiber material, which is lightweight and transparent. In some other instances, the glasses 104 are made of silica material and are transparent to provide clear vision. In all the instances, the close fitting glasses 104 are capable of delivering the heat and moisture content or medications to the eyes of the user, without obstructing the view of the user. In some other instances, the glasses 104 are made of flexible transparent material, which allows the users to easily handle the goggles 102 for storage, during wearing and during travel.

The frame structure 106 further includes one or more input means (not shown) associated with the goggles 102 for providing heat, moisture content and at least one medication to the eyes of the user. The input means of the frame structure 106 attached to the pair of protective close fitting glasses 104 leads to the number of passages for delivering the moisture content, other fluids and the medications from the diffuser 108 to the eyes of the user. In a preferred embodiment, the frame structure 106 is made from flexible synthetic materials such as rubber or polyurethane, which is comfortable for the user.

In some embodiment, the pair of protective close fitting glasses 104 is connected to the frame structure 106 using the flexible central connector to form the goggles 102 and the flexible central connector may include passages for delivering moisture content and medications to the eyes of the user. It should be understood by those skilled in the art that, the scope of present invention is not limited to shape and size of the goggle 102.

Referring back to FIG.1, the diffuser 108 in fluid communication with the frame structure 106 to provide moisture content and medications to the eyes of the user is shown. The diffuser 108 is electrically powered and may include a power cord 108c for providing an electrical connection. In some embodiment, the diffuser 108 may be battery powered. The diffuser 108 is connected to the goggles 102 using a flexible connector 110. The flexible connector 110 allows the passage of moisture contents, heat and other medications stored in the diffuser 108 to the frame structure 106 associated with the goggles 102. It is preferable to use a transparent flexible connector 110.

According to the embodiment, the diffuser 108 may include one or more mode selector means 108a, 108b configured thereon. The mode selector means 108a, 108b enable the user to select an operational mode for supplying the moisture contents, heat and other medications to the eyes of the user. In some embodiment, the mode selector means 108a, 108b may be embodied or configured on the frame structure 106 instead of on the diffuser 108. In some other embodiment, the mode selector means 108a, 108b may be provided in the form of a hand held controller. In a preferred embodiment, the mode selector means 108a, 108b is provided as push buttons or rotating switches which controls the opening of the input means for supplying saline, fluids and other medications to the eyes of the user after passing through the passages inside the frame structure 106.

The present apparatus/system 100 enables the users to select a desired mode of operation for providing a desired amount of heat, moisture and the medication to the eyes of the user for a desired period of time to treat the variety of eye conditions including dry eye syndrome or burns to the eyes. The mode selector means 108a, 108b configured on the diffuser 108 allows the users to select the operational modes including an intense mode and a standard mode for supplying desired amount of heat and moisture to the eyes of the user. The normal mode provides regular amount of heat and moisture content supply to the eyes of the user and the intense mode provides additional heat and moisture in form of saline supply to the eyes of the user.

Referring to FIG.1 and FIG. 2, as discussed above, the frame structure 106 surrounding the close fitting glasses 104 includes the input means for allowing the passage of moisture contents, heat and other medications stored in the diffuser 108, through the flexible connector 110, to the passages inside the frame structure 106. The flexible connector 110 can be removably attached to the input means of the frame structure 106.

In a preferred embodiment of the present disclosure, the input means is utilized to deliver saline to the goggles 102. The saline delivered through the goggles 102 worn by the user provides desired moisture content to the eyes of the user, thereby preventing dry eye condition of the user. In some embodiments of the present apparatus 100, the diffuser 108 stores the saline, the fluid and the medications and delivers to the goggles 102 through the input means and the passages associated with the frame structure 106 attached to the close fitting glasses 104 of the goggles 102. The diffuser 108 is electrically powered to provide saline, the fluids and the medications to the goggles 102. The saline, fluids and the medications supplied from the diffuser 108 provide heat and moisture in form of saline to the eyes of the user.

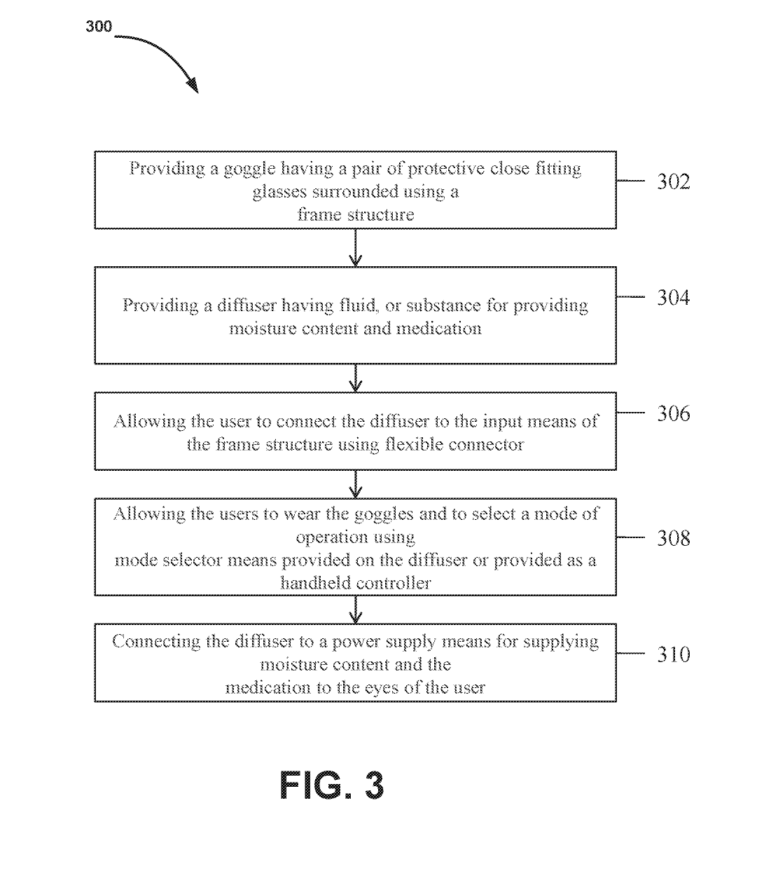

Referring to FIG. 3 shows a flowchart showing the steps for providing heat and moisture content to the eyes of the user for treating various eye conditions of a user including dry eye syndrome and/or burns to the eyes. The method starts at a step 302 that describes providing the goggle 102 having a pair of protective close fitting glasses 104 surrounded using the frame structure 106.

The method further includes a step 304 that describes providing the diffuser 108 having the fluid, saline or any other substance for providing moisture content and one or more medications.

The method further includes a step 306 that describes the user's ability to manually connect the diffuser 108 to the input means of the frame structure 106 using the flexible connector 110 having hollow interior for passing the fluid, saline or any other substance and providing moisture content and one or more medications stored in the diffuser 108. The input means (not shown) preferably includes a controllable valve, which can be controlled using the mode selector means 108a, 108a by the user. As discussed above, mode selector means 108a, 108b may be configured on the diffuser 108, or alternatively as a separate controller or configured on the frame structure 106. This allows the users to control the flow of the fluid, saline or any other substance for providing moisture content and one or more medications passing through the number of passages present inside the frame structure 106 of the goggles 102.

The method further includes a step 308 that describes the user's ability to wear the goggles 102 and can select a mode of operation using the mode selector switch or mode selector means 108a, 108b.

The method further includes a step 310 that describes connecting the diffuser 108 to a power supply means for supplying moisture content and the medication to the eyes of the user. The diffuser 108 generates steam of desired temperature and saline content and delivers through the flexible connector 110 to the frame structure 106, from where the contents are passed through the passages to the glasses 104 and around the eyes of the user. This helps to receive proper heat and moisture content around the eyes of the user and helps to treat the number of eye conditions including dry eye syndrome or burns to the eyes. The frame structure 106 of the goggles 102 is preferably made flexible and elastic to allow the users to wear for extended period of time at night and during sleeping.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

What is claimed is:

1. An apparatus 100 for treating a plurality of eye conditions of a user including dry eye syndrome or burns to the eyes comprises:
a pair of protective close fitting glasses 104 flexibly connected to form a goggle 102 to securely cover a pair of eyes of the user;
a frame structure 106 provided around the pair of protective close fitting glasses 104, wherein the frame structure 106 includes at least one input means associated with the goggles for providing heat, moisture content and at least one medication to the eyes of the user;
at least one diffuser 108 in fluid communication with the frame structure 106 to provide heat, moisture content and the at least one medication to the eyes of the user;
at least one mode selector means 108a, 108b configured on at least one of the diffuser 108, and as a hand held controller for selecting at least one desired operational mode; and
characterized in that the apparatus 100 enables the users to select a desired mode of operation for providing a desired amount of heat, moisture and the at least one medication to the eyes of the user to treat the plurality of eye conditions including dry eye syndrome or burns to the eyes.

2. The apparatus 100 of claim 1 wherein the pair of protective close fitting glasses 104 is connected using a flexible central connector to form the goggles 102, wherein the flexible central connector includes passages for delivering moisture content and at least one medication to the eyes of the user.

3. The apparatus 100 of claim 1 wherein the input means of the frame structure 106 attached to the pair of protective close fitting glasses 104 leads to a plurality of passages for passing the moisture content, at least one fluid and the at least one medication from the diffuser 108 to the eyes of the user.

4. The apparatus 100 of claim 1 wherein the input means is utilized to deliver saline to the goggles 102, wherein the saline delivered through the goggles 102 provides desired moisture content to the eyes of the user.

5. The apparatus 100 of claim 1 wherein the diffuser 108 stores the saline, the at least one fluid and the medications and delivers to the goggles 102 through the input means and the plurality of passages associated with the frame structure 106 attached to the goggles 102.

6. The apparatus 100 of claim 1 wherein the diffuser 108 is electrically powered to provide saline, the at least one fluid and the medications to the goggles 102, wherein the saline, the at least one fluid and the medications supplied from the diffuser 108 provide heat and moisture in form of saline to the eyes of the user.

7. The apparatus 100 of claim 1 wherein the mode selector means 108a, 108b allows the users to select the at least one operational mode including an intense mode and a standard mode.

8. The apparatus 100 of claim 7 wherein the intense mode provides additional heat and moisture in form of saline supply to the eyes of the user.

9. The apparatus 100 of claim 1, wherein the frame structure 106 comprising a first part 106a, and a second part 106b that engages together.

10. A method of treating a plurality of eye conditions of a user including dry eye syndrome or burns to the eyes includes the steps of:
providing a goggle 102 having a pair of protective close fitting glasses 104 surrounded using a frame structure 106,
wherein the frame structure 106 includes at least one input means for receiving moisture content and at least one medication,
wherein the frame structure 106 includes a plurality of passages for providing moisture content and at least one medication received through the input means to the eyes of the user;
providing a diffuser 108 having at least one fluid, at least one substance for providing moisture content and at least one medication;
allowing the user to connect the diffuser 108 to the input means of the frame structure 106 using at least one flexible connecting means;
allowing the users to wear the goggles 102 and to select at least one mode of operation using at least one mode selector means 108a, 108b provided configured on at least one the diffuser 108, and as a hand held controller;
connecting the diffuser 108 to a power supply means for supplying moisture content and the at least one medication to the eyes of the user; and
characterized in that the moisture content and the at least one medication provides heat and moisture content to the eyes of the user to treat the plurality of eye conditions including dry eye syndrome or burns to the eyes.

11. The method of claim 10 wherein the mode selector means 108a, 108b allows the users to select the at least one operational mode including an intense mode and a standard mode.

12. The method of claim 11 wherein selection of the intense mode provides additional heat and moisture in form of saline supply to the eyes of the user compared to standard mode.

13. The method of claim 10 wherein the frame structure 106 of the goggles 102 is flexible and elastic to allow the users to wear the goggles 102 for extended period of time at night and during sleeping.
